(19) United States
(12) Patent Application Publication
(10) Pub. No.: US 2017/0311876 A1
(43) Pub. Date: Nov. 2, 2017

(54) FORCE-MEASURING APPARATUS

(71) Applicant: SHENZHEN XIAO LUO JI TECHNOLOGY LIMITED, Shenzen (CN)

(72) Inventors: Meichen Lu, Cambridge (GB); Shan Lu, Cambridge (GB)

(73) Assignee: Shenzhen Xiao Luo Ji Technology Limited, Shenzen (CN)

(21) Appl. No.: 15/525,146

(22) PCT Filed: Nov. 11, 2015
(86) PCT No.: PCT/IB2015/002094

§ 371 Date: May 8, 2017

(30) Foreign Application Priority Data
Nov. 11, 2014 (GB) 1420047.1

(51) Int. Cl. A61B 5/00; A61B 5/11; A61B 5/22; A61B 90/00

(52) U.S. Cl. CPC A61B 5/4585; A61B 5/6812; A61B 5/112; A61B 5/221; A61B 5/7475; A61B 5/746; A61B 5/486; A61B 2090/064; A61B 2503/10; A61B 2562/0219

(57) ABSTRACT

The invention relates to apparatus to be worn at or adjacent to the knee joint of a person to detect the impact effect of forces applied to the knee joint and, as a result of the detection, and typically analysis of information relating to the person wearing the apparatus, feedback information is provided to the person, in one embodiment during the time when the apparatus is being worn, so as to provide timely feedback to the person wearing the apparatus of the impact on the PF joint. The person wearing the apparatus or monitoring the same can use the feedback whilst the apparatus is being worn, in order to allow the person to change their movement or stop their movement with immediate effect and thereby prevent or minimise the risk of the injury occurring.

The invention to which this application relates is a force-measuring apparatus. Specifically, but not necessarily exclusively, the present invention relates to an apparatus for use in the detection of excessive force applied on the patellofemoral joint (PF joint) of the knee of a person.

The knee is subject to complex forms of stress and the patellofemoral joint is often particularly susceptible to the stress. It is known that the pf joint can be injured during sporting activities due to one off occurrences or due to repeated stress or impact. For example, patellar tendinitis (“jumper's knee”) is a painful condition that may result from the person repetitively running and/or jumping.

The injury to the pf joint can result from overstress during the act of running and/or jumping and/or due to the fact that the peak compressive force applied to the pf joint during these activities is shown to be 10-14 times the body weight (Stephen, et al. 2008, Flynn and Soutas-Little 1995). There is a positive correlation between relatively large and rapid impact forces and the overuse and injury of the knee joint through running. A number of researches have suggested that runners with relatively large and rapid impact forces are at an increased risk of developing an overuse injury of the lower extremity. Hreljac, in the paper “Hreljac, A., 2004. Impact and overuse injuries in runners. Medicine and science in sports and exercise, 36(5), pp. 845-849.” reviewed the causes of overuse running injuries and emphasized on the effect of impact forces.

By comparing injury free runners with a group who had sustained at least one overuse running injury, the biomechanical variables that are significantly lower for injury free group were vertical impact peak and maximal vertical loading rate as identified in the paper by “Hreljac, A., Marshall, R. N. & Hume, P. a, 2000. Evaluation of lower extremity overuse injury potential in runners. Medicine and science in sports and exercise, 32(48), pp. 1635-1641”.

In the paper by “Messier, S. P. et al., 2008. Risk factors and mechanisms of knee injury in runners. Medicine and Science in Sports and Exercise, 40(14), pp. 1873-1879” it is suggested that such evidence indicated that high forces applied to the lower extremity tissues may be mechanisms of knee injuries and linked the behavioural (training history, injury history), in addition to physiological risk factors with potential underlying mechanisms of injury. Similar evidence is provided in the paper by “Lun, V. et al., 2004. Relation between running injury and static lower limb alignment in recreational runners. British journal of sports medicine, 38(VI), pp. 576-580.” where subjects with mild knee pain had a 37% higher loading rate of vertical ground reaction force.

Furthermore the application of excessive normal forces can result in overuse syndromes by high repetition low loads or low repetition high loads (Davies, Wallace and Malone 1980).

In addition to the application of forces, a number, and possible combination, of factors, including misalignment of feet and/or improper gait, such as overstriding and understriding, can lead to high loads being applied (Davies, Wallace and Malone 1980) and/or the affect of the loads being exacerbated. Studies have shown that lower limb alignment contributes to running injuries (Lun, et al. 2004), which can be determined by measuring the direction of the impact.

The concept of protecting the patellofemoral joint (PF joint) with a band or strap structure is known. For example, U.S. Pat. No. 7,594,897 provides a means of applying local pressure to reduce knee pain; U.S. Pat. No. 6,852,088 discloses an adjustable device which provides radial pressure and U.S. Pat. No. 7,730,550 and U.S. Pat. No. 5,865,782.

Devices can also be provided which measure foot impacts with appropriate sensors at the users' shoes, for example, as described in U.S. Pat. No. 8,581,731 but this device is unable to determine the impact at the knee, or the undesirable direction of the impact. US20120259255 discloses a device that assists patients suffering from peripheral neuropathy to restore normal gait.

It is therefore an aim of the present invention to provide a wearable apparatus that measures deceleration/impact at the knee of the wearer and/or the direction of said deceleration/impact. It is envisaged that this will be particularly useful in the prevention or minimising of injury to persons when performing sporting activities.

According to a first aspect of the invention there is provided an apparatus for detecting and measuring one or more forces exerted on, or adjacent to, a joint of a person's knee, wherein said apparatus includes a sensing means for detecting and measuring at least one property of said exerted force; power supply means; and data processing means connected to, or included with, the said sensing means which is worn by the user at, or adjacent to, the said knee during use and wherein the apparatus provides feedback information to the person and/or another person in relation to the measured force and/or the impact effect exerted on the said knee joint.

Typically the feedback information is provided to the person during the time when the apparatus is being worn so as to provide timely feedback to the person wearing the apparatus of the impact on the PF joint. This therefore means that the person wearing the apparatus or monitoring the same is provided with feedback which can be used whilst the apparatus is being worn, in order to allow the person to change their movement at that time to reduce the risk of injury or, if the feedback is that the person is at immediate risk, the person can stop their movement with immediate effect and thereby prevent the injury from occurring.

In one embodiment the feedback includes information provided to assist in the user taking preventative action to reduce the risk of knee injury.

In one embodiment, the apparatus includes data transfer means to allow the feedback information to be provided.

In one embodiment said data transfer means permit transfer of data to and from the apparatus. Thus, in one embodiment, the apparatus can be input with specific information relating to the user prior to use. Such information may relate to the weight, height etc. of the user. Consequently, the apparatus may better calculate the impact of forces exerted on the user and detect when a predetermined threshold is reached and beyond which damage to the knee joint may be caused.

In one embodiment, said data transfer means may be wired and/ or wireless. Typically, wireless data transfer means include, but are not limited to, Wireless radio frequency (RF), Wi-Fi, Bluetooth and/or the like.

In one embodiment, said data processing means calculate the impact of said one or more forces exerted on the knee joint of the user. Typically, the data processing means determines whether the impact of a force exerted on the user exceeds a predetermined maximum force that is to be exerted. Further typically, said predetermined force is calculated based on specific information relating to the user that has been input on the apparatus.

In one embodiment, the apparatus further includes alert means. Typically, said alert means are provided in the form of one or more of the following: audible alert means; visual alert means; and/or motional alert means. Further typically, said alert means activate when the forces exerted on a knee of the user exceed a predetermined threshold.

In one embodiment, the apparatus further includes a reminder system programmed therein. Typically, said reminder system generates customised feedback to the user based on user action. Further typically, said feedback may be provided to either affirm the correct running gait adopted by the user, or warn the user of the danger of a force exerted beyond a predetermined threshold.

In one embodiment the sensing means is located with a strap which is selectively positioned at or adjacent to the person's knee.

In one embodiment the sensing means is provided at a predetermined location with respect to the strap, and hence the persons knee, which is to be monitored.

In one embodiment, the strap is provided in the form of an elasticated band of a size so as to be suitable to be worn around the knee joint of the user.

In one embodiment, the sensing means is a three-axis accelerometer. Typically, the accelerometer is provided to measure the amplitude and the direction of the forces exerted on the knee joint of a user as the apparatus is worn. In one embodiment the accelerometer is a three-axis microelectromechanical (MEMS) accelerometer.

In one embodiment, said sensing means detects and measures at least two forces when exerted on the user along orthogonal axes. Consequently, the apparatus can evaluate impacts/forces exerted laterally on the user's joint.

In one embodiment, said apparatus extends substantially around the knee joint of the user. Typically, said apparatus is located just above or just below the knee joint of a user.

In one embodiment, a plurality of sensing means are provided in the apparatus and are located at spaced location substantially around the knee joint of a user, when the apparatus is worn.

In one embodiment, said data processing means is provided in the form of an integrated circuit and typically includes a micro controller unit (MCU). Further typically, said MCU includes sensor, memory, and timer circuitry.

In one embodiment, said power supply means are provided in the form of one or more batteries. Typically, said power supply means is rechargeable. Typically, said power supply means provides power to the other components within the apparatus.

In one embodiment, said apparatus is connectable to an external power supply.

In one embodiment the said joint of the knee which is monitored is the patellofemoral joint.

In a further aspect of the invention there is provided a method of monitoring the force exerted on at least one joint of a knee, said method comprising the steps of mounting a three-axis sensing means via a mounting means at or adjacent to the said knee joint, to measure the amplitude and direction of forces applied to the said joint, transmitting data via data processing means which is representative of the said measured force, calculating the impact effect of the measured forces exerted on the said joint with reference to information specific to the person whose knee is being monitored and if the calculation indicates that the impact effect exceeds a predetermined threshold value an alert is generated.

Typically the alert can be any, or any combination, of audible (speaker), visual (LED) and/or a motional (vibrator) alert.

In one embodiment the information relating to the person can be provided via a wireless radio frequency (RF) transmitter from a mobile peripheral device (105).

In one embodiment at least part of the information is used to assess the force exerted on the person above the said joint.

Thus, the apparatus of the present invention provides a wearable device that contains sensing means which measure the direction and amplitude of one or more forces exerted on, for example, at least one joint of the knee of a user during running exercise and footwork, and consequently provides feedback based on previously input information of weight, height etc. of the user. The information is processed by data processing means within the apparatus and analysed in relation to personal data of the user to provide customised feedback. The feedback can be presented in various forms such as instant notification and wireless transmission of data for further analysis, in order to help the user to attain or maintain the desired running gait and footwork to avoid knee injury.

Embodiments of the present invention will now be described with reference to the accompanying figures, wherein:

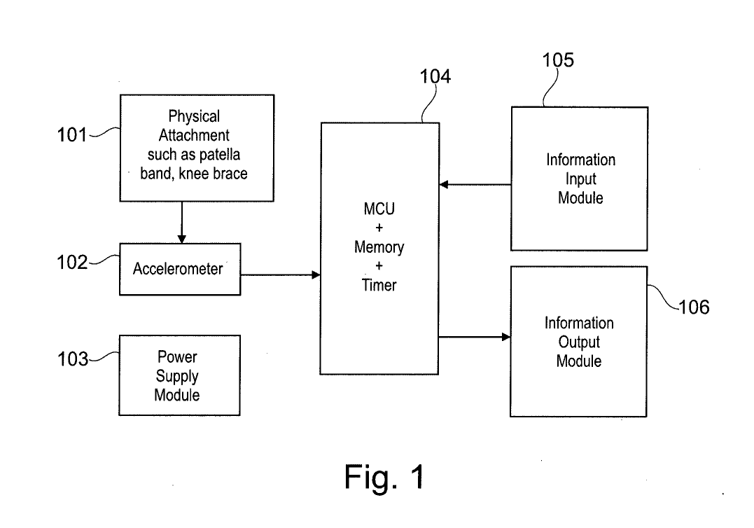

FIG. 1 shows a flow diagram of the method of operation of the apparatus in accordance with an embodiment of the present invention;

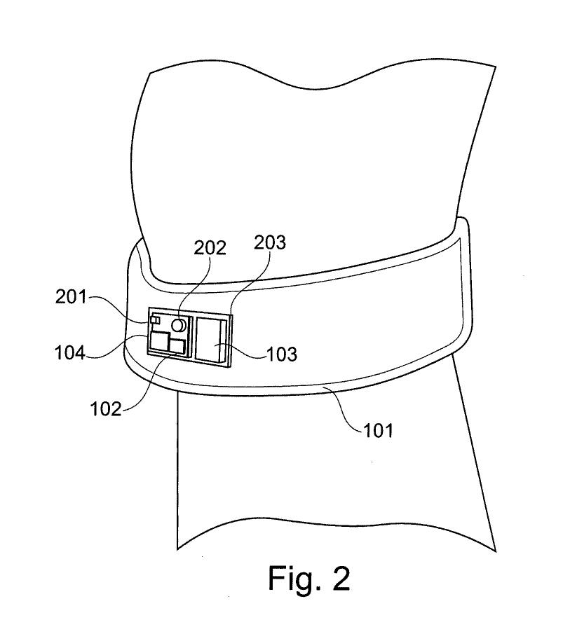

FIG. 2 shows an electronic component of an apparatus in accordance with an embodiment of the present invention;

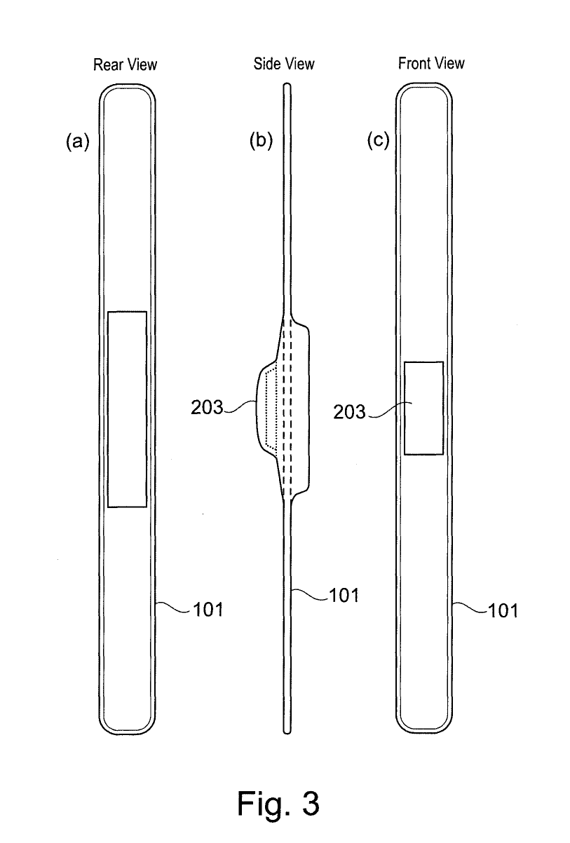

FIGS. 3a- c show the (a) rear view, (b) side view and (c) front view of an apparatus in accordance with an embodiment of the present invention;

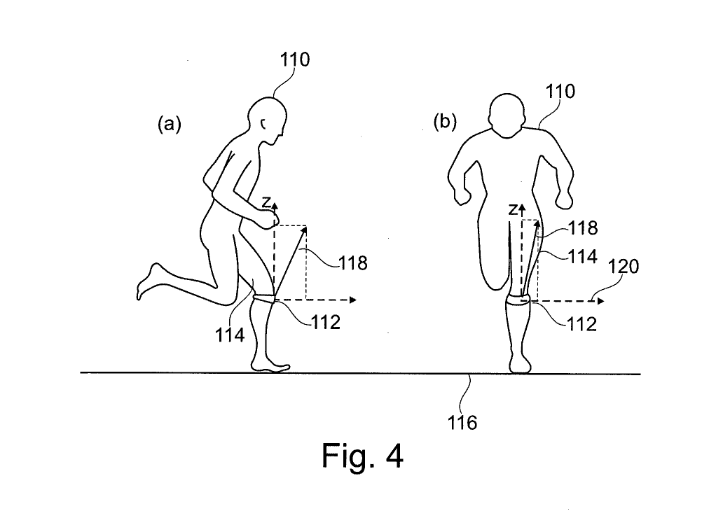

FIGS. 4a- b describe the measurement of impact effect on (a) a person running in side view and (b) a person running in front view and wearing an apparatus in accordance with an embodiment of the present invention; and

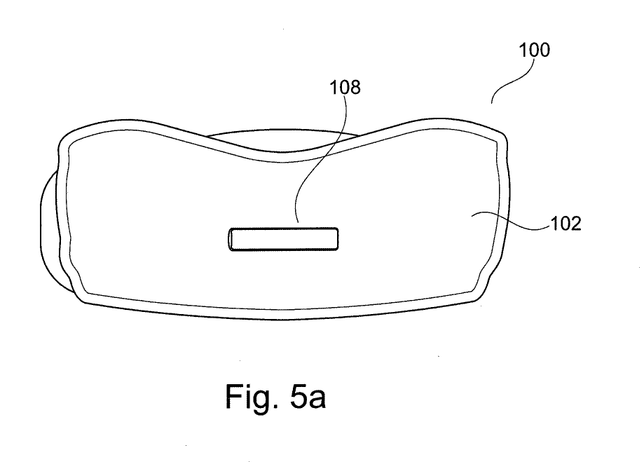

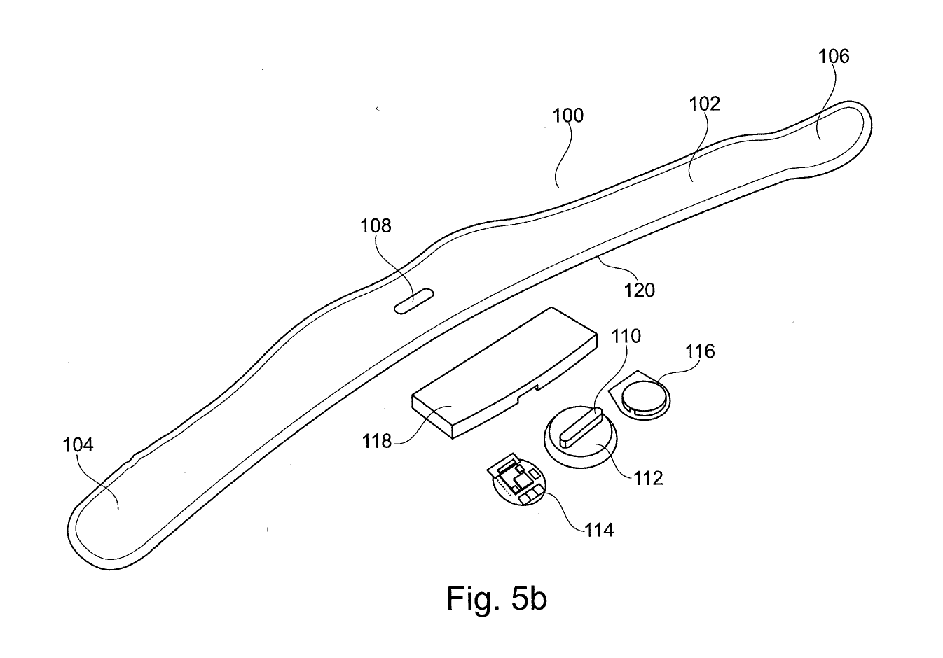

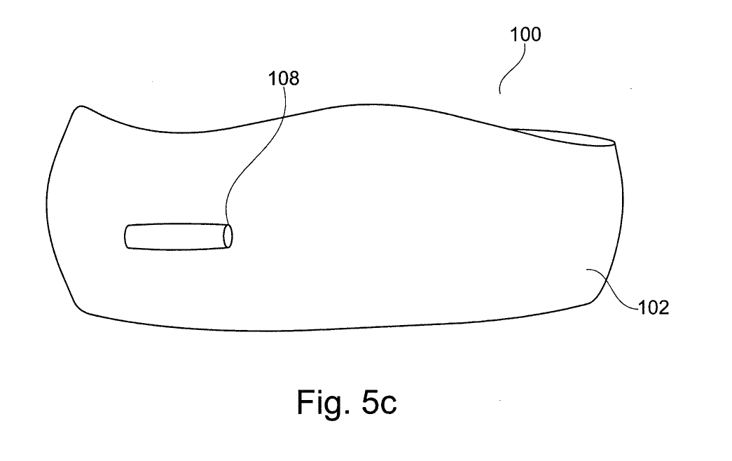

FIGS. 5a-c illustrate an embodiment of apparatus in accordance with the invention.

Referring firstly to FIG. 1, the flow diagram demonstrates the method workflow in accordance with the present invention. There is provided sensing means in the form of a three-axis accelerometer (102) provided to be mounted with respect to the joint of a person via a physical attachment (101), which, most typically, is provided in the form of a knee band to be worn by a person either on, just below or just above the knee joint which is to be monitored.

The accelerometer measures the force amplitude and direction which his impacted on the knee joint with respect to which the apparatus is mounted. Data representative of the measured force is transmitted via data processing means in the form of an integrated circuit (104) which includes a memory, timer and Micro Controller Unit (MCU). The power supply (103) supplies power to the electronic timer/controller integrated circuits and MCU. The MCU calculates the impact effect of the various forces which are detected by the accelerometer 102 as having been exerted on the knee joint.

If the impact effect on the joint, such as the patellofemoral joint (PF joint), is calculated to have exceeded a predetermined threshold then action can be taken.

Typically the impact effect is calculated with reference to information which is specific to the person, such as their weight, height, heart rate, BMI and other physical and/or social and/or environmental factors. This information is typically input to the MCU prior to and/or during the wearing and use of the apparatus via module 105.

In the event that the predetermined threshold value is exceeded by the calculated impact effect then one of the actions can be the generation of an alert by an information output module (106), and the alert can be any or any combination of audible (speaker), visual (LED) and/or a motional (vibrator) alert.

The information to and from the respective modules 105, 106 can be transmitted via a wireless radio frequency (RF) transmitter (e.g. Bluetooth) from a mobile peripheral device (105). In one embodiment part of the information is used to assess the force exerted above the PF joint. Additionally, the information can be stored in memory means provided in the mobile peripheral (106) and is accessible over a period of time as a tool for tracking the history of the impact effect and alerts generated for the person wearing the apparatus.

FIG. 2 illustrates the mounting means (101) in the form of a knee strap and/or a belt adapted to encircle the leg at the region of the knee. Typically the strap has sufficient elasticity or is sufficiently tight to thereby retain the same in position. Electronic components of the apparatus such as the power supply means, data processing means, alert means and information output and input modules (203) are located at the front part of the strap when worn. The accelerometer (102), in the form of a three-axis accelerometer to detect direction and amplitude of the force exerted on the PF joint is provided on the strap. Additional accelerometers and/or other sensing components may be located within the strap (101) at points located around the knee joint. The power supply contains battery (103) to supply power to the electronic circuits. In addition, an external power supply with an AC to DC converter may also be provided.

In order to notify the user if and when a predetermined impact effect threshold value is met or exceeded, the alert means can be operated in one or a combination of various forms: a motor (202) generates a vibration to alert the user and, additionally, a wireless alert signal may be provided. The alert is transmitted to a peripheral device, e.g. mobile phone or Bluetooth headphone and an audible alert may be activated in the form of a beep, alarm etc.

Within the integrated circuit (104) of the apparatus, the MCU implements algorithms to process input information and generate output information. Also included can be a sensor input circuit; timer circuitry; a memory circuit; an alert function, driver circuit for a vibrator, speaker etc.; and wireless radio frequency (RF) transmitter circuitry to allow remote information access are also included.

Referring to FIGS. 3a-c, the mounting means (101) is shown as being adapted to encircle the leg at the PF joint region, and therefore may encircle the top and/or bottom part of the joint. The mounting means can include a buttress support at the rear when it is worn. The electronic components (203) are shown located with the mounting means (101) at a fixed location, although this need not be restricted to the centre of the belt (101) facing outward, as shown in the present figures.

FIG. 4 describes the measurement of the effect of impact by forces on a person's 110 knee joint 112 on their leg 114 at a side view (FIG. 4a) and a front view (FIG. 4b) as they are running along a surface 116. In the case of normal running gait, the magnitude of the impact effect, which is proportional to the acceleration upon impact (shown as the solid arrow 118), is the major factor that causes damage and/or pain to the knee joint. The horizontal component of the force 120 in the x-axis corresponds to sideway force imparted on the PF joint 112, which is the cause of misalignment that, in turn, contributes to knee damage.

FIGS. 5a-c illustrate apparatus 100 in a form to be worn. The apparatus includes a strap 102 which in this case has a hook and loop fastening arrangement so as to allow the ends 104-106 of the same to be brought into engagement at a location which allows the strap to be closely fitted in position around the person's leg at or adjacent to the knee joint, and be retained in that position during use.

The strap includes an aperture 108 in which is located the sensing means 110 in the form of an accelerometer. Although in this example there is only provided one sensing means it should be appreciated that in other embodiments a plurality of accelerometers can be provided at spaced locations. The accelerometer 110 is mounted on a support 112 and the accelerometer 110 is placed into the apertures 108 to be retained therein.

The support 112, processing means circuit 114 and power supply 116 are all located on a backing layer 118 which is attached to the inner surface 120 of the strap so that the same are effectively sandwiched between the strap 102 on one side and the backing layer 118 on the other side and thereby protected from damage when being worn.

Thus, the apparatus of the present invention provides a wearable device that contains a number of sensing means which measure the direction and amplitude of one or more forces exerted on, for example, the knee of a user during running exercise and footwork, and consequently provides feedback based on previously input information of weight, height etc. of the user. The information is processed by data processing means within the apparatus and analysed in relation to personal data of the user to provide customised feedback. The feedback can be presented in various forms such as instant notification and wireless transmission of data for further analysis, in order to help the user to attain or maintain the desired running gait and footwork to avoid knee injury.

1: An apparatus for detecting and measuring one or more forces exerted on, or adjacent to, a joint of a person's knee, wherein said apparatus includes a sensing means for detecting and measuring at least one property of said exerted force; power supply means: and data processing means connected to, or included with, the said sensing means which is worn by the user at, or adjacent to, the said knee during use, the apparatus
provides feedback information to the person and/or another person in relation to the measured force and/or the impact effect exerted on the said knee joint wherein the apparatus includes a reminder system to generate feedback to confirm when a correct gait is adopted by the user or warn of the danger of the user's gait causing the force impact effect to be beyond a predetermined threshold value

2: Apparatus according to claim 1 wherein the feedback includes information provided to assist in the person taking preventative action to reduce the risk of injury to the said knee joint.

3: Apparatus according to claim 1 wherein the apparatus includes data transfer means to allow the feedback information to be provided.

4: Apparatus according to claim 3 wherein said data transfer means permit transfer of data to and from the apparatus.

5: Apparatus according to claim 1 wherein the feedback information is provided to the person during the time when the apparatus is being worn and is in use.

6: Apparatus according to claim 5 wherein the feedback information provided when the apparatus is being worn allows the person to change their movement at that time to reduce the risk of injury or stop their movement with immediate effect and thereby prevent or minimise the risk of injury occurring.

7: Apparatus according to claim 1 wherein specific information relating to the person using the apparatus is input to the apparatus prior to and/or during use.

8: Apparatus according to claim 7 wherein the said information is referred to in the calculation of the impact effect of forces measured as being exerted on the said knee joint.

9: Apparatus according to claim 1 wherein the calculated impact effect is compared to a predetermined threshold value beyond which damage to the knee joint may be caused.

10: Apparatus according to claim 1 wherein the apparatus includes alert means which activate when the calculated impact effect matches or exceeds the predetermined threshold value.

11. (canceled)

12: Apparatus according to claim 1 wherein the sensing means is located on a strap which is selectively positioned at or adjacent to the person's knee.

13: Apparatus according to claim 12 wherein the sensing means is provided at a predetermined location with respect to the strap, and hence the person's knee, when worn.

14: Apparatus according to claim 1 wherein the sensing means is a three-axis accelerometer.

15: Apparatus according to claim 1 wherein said sensing means detects and measures at least two forces when exerted on the user along orthogonal axes.

16: Apparatus according to claim 15 wherein the apparatus evaluates the impact effect of forces exerted laterally on the knee joint.

17: Apparatus according to claim 1 wherein a plurality of sensing means are provided in the apparatus and are located at spaced locations substantially around the knee joint of a user, when the apparatus is worn.

18: Apparatus according to claim 1 wherein the data processing means include a micro controller unit (MCU), memory, and timer.

19: Apparatus according to any of the preceding claims wherein the said knee joint is the patellofemoral joint.

20: A method of monitoring the force exerted on at least one joint of a knee, said method comprising the steps of mounting a three-axis sensing means via a mounting means at or adjacent to the said knee joint, to measure the amplitude and direction of forces applied to the said joint, transmitting data via data processing means which is representative of the said measured force, calculating the impact effect of the measured forces exerted on the said joint with reference to information specific to the person whose knee is being monitored and generating feedback to confirm when a correct gait is adopted by the user or to alert of the danger of the users gait causing the force impact effect to be beyond a predetermined threshold value if the calculation indicates that the impact effect exceeds a predetermined threshold value.

21: A method according to claim 20 wherein the alert is any, or any combination, of audible (speaker), visual (LED) and/or a motional (vibrator) alert.

22: A method according to claim 21 wherein the information relating to the person is provided via a wireless radio frequency (RF) transmitter from a mobile peripheral device (105).

23: A method according to claim 20 wherein the sensing means measure the direction and amplitude of one or more forces exerted.
